(19) United States
(12) Patent Application Publication
(10) Pub. No.: US 2014/0046068 A1
(43) Pub. Date: Feb. 13, 2014

(54) METHOD OF SYNTHESIZING BEPOTASTINE OR BENZENESULFONIC ACID SALT THEREOF AND INTERMEDIATES USED THEREIN

(71) Applicant: EVERLIGHT USA, INC., Pineville, NC (US)

(72) Inventors: Tsung-Ting CHEN, Taoyuan County (TW); Hao-Cheng YANG, Taoyuan County (TW); Tsai-Yung CHOU, Taoyuan County (TW); Chi-Hsiang YAO, Taoyuan County (TW)

(73) Assignee: EVERLIGHT USA, INC., Pineville, NC (US)

(21) Appl. No.: 13/758,065

(22) Filed: Feb. 4, 2013

(30) Foreign Application Priority Data
Aug. 10, 2012 (TW) 101128942

(51) Int. Cl. C07D 401/12

(52) U.S. Cl. CPC C07D 401/12
USPC 546/194

(57) ABSTRACT

The present invention relates to a novel method of synthesizing bepotastine or its benzenesulfonic acid salt and novel intermediates used therein. The present invention uses L-α-hydroxy acid for chiral resolution to form an L-α-hydroxy acid salt of a compound represented by the following formula (VII-1), so as to synthesize bepotastine or its benzenesulfonic acid salt in high optical purity.

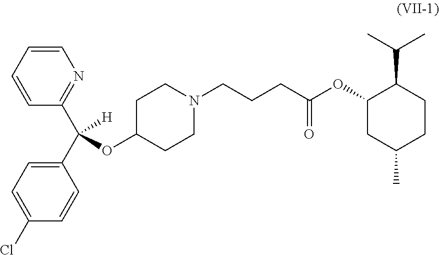 [SMILES: CC(C)[C@H]1CC[C@H](C)C[C@@H]1OC(=O)CCCN1CCC(O[C@@H](c2ccc(Cl)cc2)c2ccccn2)CC1]

### CROSS REFERENCE TO RELATED APPLICATION

This application claims the benefits of the Taiwan Patent Application Serial Number 101128942, filed on Aug. 10, 2012, the subject matter of which is incorporated herein by reference.

### BACKGROUND OF THE INVENTION

1. Field of the Invention

The present invention relates to a novel method of synthesizing bepotastine or benzenesulfonic acid salt thereof and novel intermediates used therein.

2. Description of Related Art

Bepotastine besilate is an organic salt of bepotastine and benzenesulfonic acid, and can be used as a selective and rapidly effective antihistamine for the treatment of allergic diseases such as allergic rhinitis, allergic conjunctivitis etc. It has been reported that (S)-bepotastine has better antihistaminic activity and anti-allergic activity compared to (R)-bepotastine. Therefore, it is an important issue for the preparation of bepotastine and its benzenesulfonic acid to obtain the (S)-isomer in high purity.

As disclosed in U.S. Pat. No. 6,780,877, the (S)-isomer for synthesis of a desired final compound can be obtained by optically resolving (RS)-4-[(4-chloro phenyl)(2-pyridyl) methoxy]piperidine using (2R,3R)-2-hydroxy-3-(4-methoxyphenyl)-3-(2-nitro-5-chlorophenylthio) propionic acid. Further, in U.S. Publication No. 2010/0168433, N-benzyloxycarbonyl L-aspartic acid was applied in the optical resolution of (RS)-bepotastine L-menthyl ester derived from (RS)-4-[(4-chlorophenyl)(2-pyridyl) methoxy]piperidine to obtain the (S)-bepotastine for synthesis of a desired final compound. However, these asymmetric acids applied in the two aforementioned methods cannot be obtained unless synthesized, and thus have the disadvantages of non-ready availability and high price.

Accordingly, the object of the present invention is to develop a safe, effective and low-cost method of preparing bepotastine and its benzenesulfonic acid salt in high optical purity.

### SUMMARY OF THE INVENTION

The object of the present invention is to provide a novel method of synthesizing bepotastine or its benzenesulfonic acid salt, in which bepotastine and its benzenesulfonic acid salt can be prepared in high optical purity by a safe, effective and low-cost synthetic route.

To achieve the object, the present invention provides a method of preparing bepotastine or its benzenesulfonic acid salt, which includes following steps:

(a) providing a compound represented by the following formula (V),

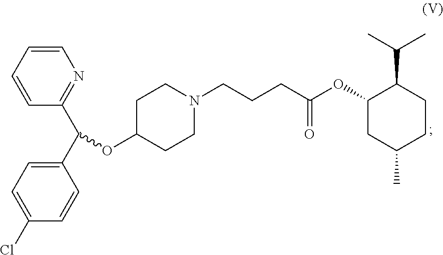 [SMILES: CC(C)[C@H]1CC[C@H](C)C[C@@H]1OC(=O)CCCN1CCC(OC(c2ccc(Cl)cc2)c2ccccn2)CC1]

(b) treating the compound of the formula (V) with L-α-hydroxy acid to conduct optical resolution process and isolating L-α-hydroxy acid salt formed of a compound represented by the following formula (VII-1) and the L-α-hydroxy acid,

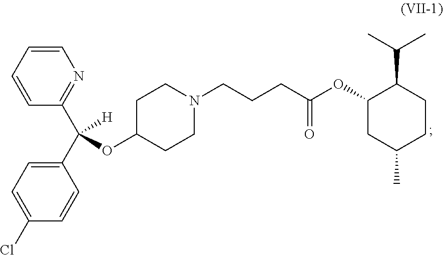 [SMILES: CC(C)[C@H]1CC[C@H](C)C[C@@H]1OC(=O)CCCN1CCC(O[C@@H](c2ccc(Cl)cc2)c2ccccn2)CC1]

and

(c) subjecting the L-α-hydroxy acid salt to acid removal and ester hydrolysis treatment to produce a compound represented by the following formula (VIII) or to further nroduce benzenesulfonic acid salt thereof,

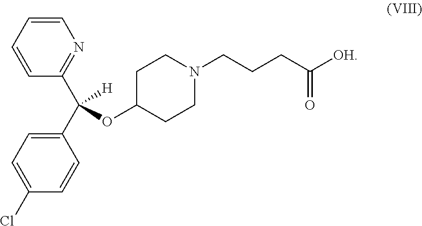 [SMILES: O=C(O)CCCN1CCC(O[C@@H](c2ccc(Cl)cc2)c2ccccn2)CC1]

In the present invention, examples of L-α-hydroxy acid can include L-tartaric acid, L-mandelic acid, L-malic acid, dibenzoyl-L-tartaric acid, di-p-toluoyl-L-tartaric acid and so on. However, L-tartaric acid is preferred, and the compound represented by the following formula (VI) can be produced,

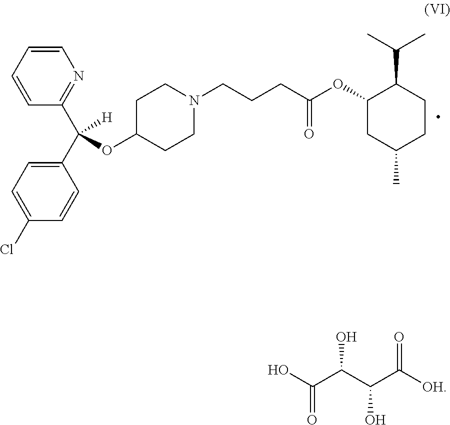 [SMILES: CC(C)[C@H]1CC[C@H](C)C[C@@H]1OC(=O)CCCN1CCC(O[C@@H](c2ccc(Cl)cc2)c2ccccn2)CC1 ; O=C(O)[C@H](O)[C@@H](O)C(=O)O]

Accordingly, the present invention can isolate the desired stereoisomer by using D-menthyl ester as a chiral auxiliary and using L-α-hydroxy acid for optical resolution to significantly enhance the optical purity. For example, in the step (b), the L-tartaric acid salt can be produced in optical purity of larger than 99% (S-isomer/S+R isomers). Since L-tartaric acid, L-mandelic acid and L-malic acid used in the present invention are naturally available, the method of the present invention can significantly reduce the manufacturing cost compared to the methods disclosed in U.S. Pat. No. 6,780,877 and US 2010/0168433, without the shortcomings of requirement for self-directed synthesis of specific asymmetric acids and high production cost.

In the present invention, the step (b) can include: mixing the compound of the formula (V) and the L-α-hydroxy acid in a first organic solvent to produce the L-α-hydroxy acid salt; and conducting recrystallization of the L-α-hydroxy acid salt with a second organic solvent and isolating crystals of the L-α-hydroxy acid salt.

In the present invention, the amount of L-α-hydroxy acid used for the chiral resolution can be about 0.5 to 2.0-fold moles based on the compound of the formula (V). With regard to the step of producing L-α-hydroxy acid salt, the reaction temperature can range from about 0 to 75° C., and the reaction time can range from about 1 to 50 hours.

In the present invention, the first organic solvent used in the optical resolution process is not particularly limited as long as no adverse effect on optical resolution occurs. For example, in accordance with one embodiment of the present invention, the optical resolution can be carried out in a mixed solvent of acetone and n-hexane, but it is not limited thereto.

In the present invention, the second organic solvent used in recrystallization of the L-α-hydroxy acid salt is not particularly limited, and one skilled person in the art can select an appropriate solvent for recrystallization. For example, in accordance with one embodiment of the present invention, the recrystallization of L-tartaric acid salt represented by the above formula (VI) can be carried out in a mixed solvent of acetone and ethanol, but it is not limited thereto.

In the present invention, the L-α-hydroxy acid salt produced by the optical resolution process can be collected by various separation methods, such as filtration, centrifugation, and others.

The ester hydrolysis in the above step (c) is not particularly limited, and one skilled person in the art can select an appropriate agent (such as alkaline liquor) and reaction condition to convert ester group into carboxyl group according to ordinary knowledge, namely hydrolyzing the compound (VII-1) into the compound (VIII).

In the present invention, the compound of the formula (V) can be prepared by the following step: subjecting a compound represented by the following formula (III) and a compound represented by the following formula (IV) to substitution reaction, thereby producing the compound of the formula (V),

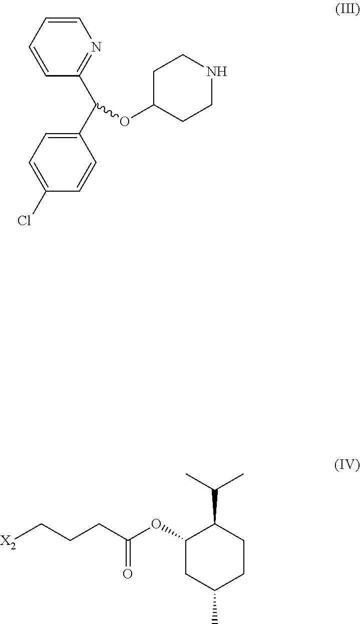

wherein X₂ is a leaving group.

The compound of the formula (III) can be produced by the following steps: subjecting a compound represented by the following formula (I) and a compound represented by the following formula (II) to substitution reaction and then deprotection reaction, thereby producing the compound of the formula (III),

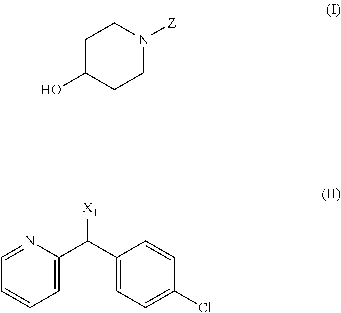

wherein Z is a protecting group for amino group, and X₁ is a leaving group. Herein, the substitution reaction of the compounds (I) and (II) can be conducted in the absence of solvent, thereby reducing the use amount of solvent.

In the compound (I) of the present invention, Z is not particularly limited, and one skilled person in the art can select an appropriate protecting group for amino group according to ordinary knowledge. However, it is preferable to use acyl group (such as acetyl group) as the protecting group for amino group, such that the deprotection process can be simplified into a one-pot process by, for example, pouring acid liquor or alkaline liquor thereto.

In the compounds (II) and (IV) of the present invention, X₁ and X₂ are not particularly limited, and one skilled person in the art can select an appropriate leaving group for substitution reaction according to ordinary knowledge. For example, in accordance with one embodiment of the present invention, the leaving group can be halogen (such as F, Cl, Br, I), but is not limited thereto.

Moreover, the compound (III) can be purified before being subjected to the reaction of producing the compound (V) by the following steps: producing and isolating a salt compound represented by the following formula (III′) using dibenzoyl-DL-tartaric acid, and then conducting acid removal treatment to liberate the compound (III),

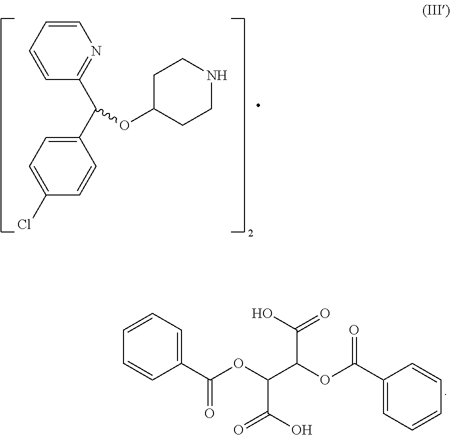

Accordingly, the oily compound (III) that is difficult to be purified can be converted into a solid salt, and then the solid salt that is easy to be stored can be obtained in high purity by simple separation method. Subsequently, the compound (III) can be achieved in high purity of larger than 95% by acid removal treatment.

In the present invention, the term “acid removal” refers to a process of removing acid molecule from a salt compound. For example, the dibenzoyl-DL-tartaric acid of the salt compound (III′) can be removed using an alkaline liquor to obtain the compound (III). Similarly, L-tartaric acid of the salt compound (VI) also can be removed using an alkaline liquor to obtain the compound (VII-1).

The method of the present invention can further include a step: recovering a compound of the following formula (VII-2) which is excluded in the optical resolution process, and converting the compound (VII-2) to the compound (V),

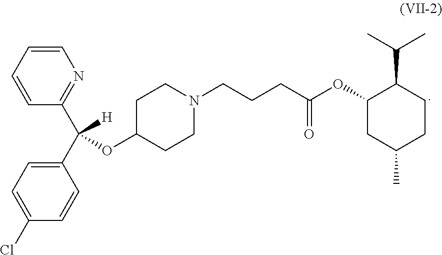 [SMILES: CC(C)[C@H]1CC[C@H](C)C[C@@H]1OC(=O)CCCN1CCC(O[C@H](c2ccc(Cl)cc2)c2ccccn2)CC1]

In accordance with the present invention, the compound (V) obtained by the recovering and converting treatments can be further subjected to the steps (a)-(c) so as to get the desired product. By repeating the above recovering and converting treatments, lots of raw materials and reagents can be saved.

In more detail, after conducting optical resolution and isolating the salt compound (VI), the mother liquor from which the salt compound (VI) is isolated can be recovered and treated with a base to produce the compound (VII-2), followed by the converting reaction. Herein, the converting reaction is not particularly limited, and one skilled person in the art can select appropriate reagents (such as acid) and reaction condition to convert the compound (WI-2) into the racemic mixture represented by the formula (V) according to ordinary knowledge. For example, in accordance with one embodiment of the present invention, acetic acid can be used to conduct the converting step. However, it is not limited thereto.

Furthermore, the compound (VIII) obtained by the present invention can be further reacted with benzenesulfonic acid to produce benzenesulfonic acid salt represented by the following formula (IX),

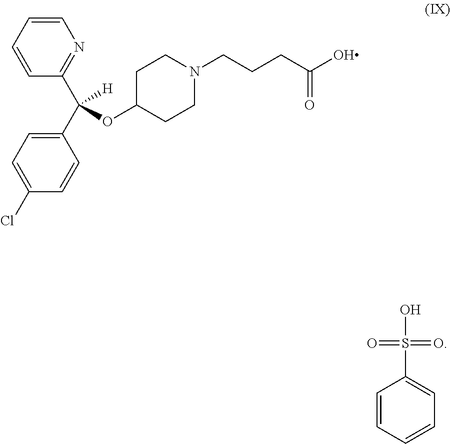

In the present invention, one skilled person in the art can produce the benzenesulfonic acid salt (IX) (i.e. bepotastine besilate) by conventional methods, and the purity of bepotastine besilate can be enhanced by recrystallization. The recrystallization of the benzenesulfonic acid salt is not particularly limited, and one skilled person in the art can select an appropriate solvent to carry out recrystallization. For example, in accordance with one embodiment of the present invention, a mixed solvent of acetone and water can be used in recrystallization of the benzenesulfonic acid salt, thereby obtaining bepotastine besilate with optical purity of larger than about 99.9%.

In conclusion, the present invention can isolate the desired stereoisomer using readily available L-α-hydroxy acids to conduct optical resolution and produce novel L-α-hydroxy acid salt intermediates, such that bepotastine or its benzenesulfonic acid salt can be synthesized in high optical purity.

### BRIEF DESCRIPTION OF THE DRAWINGS

FIG. 1 shows an X-ray diffraction pattern of the compound (III′) according to the present invention;

FIG. 2 shows an X-ray diffraction pattern of the compound (VI) according to the present invention; and

FIG. 3 shows an X-ray diffraction pattern of the compound (IX) according to the present invention.

### DETAILED DESCRIPTION OF THE PREFERRED EMBODIMENT

Hereafter, examples will be provided to illustrate the embodiments of the present invention. Other advantages and effects of the invention will become more apparent from the disclosure of the present invention. Other various aspects also may be practiced or applied in the invention, and various modifications and variations can be made without departing from the spirit of the invention based on various concepts and applications.

The below examples of the present invention provide a safe, effective and low-cost synthetic pathway, in which purity can be enhanced by the formation of solid salts, thereby obtaining bepotastine and its benzenesulfonic acid salt in high optical purity.

The following synthetic scheme 1 shows a new synthetic pathway of bepotastine besilate in accordance with one preferred example of the present invention.

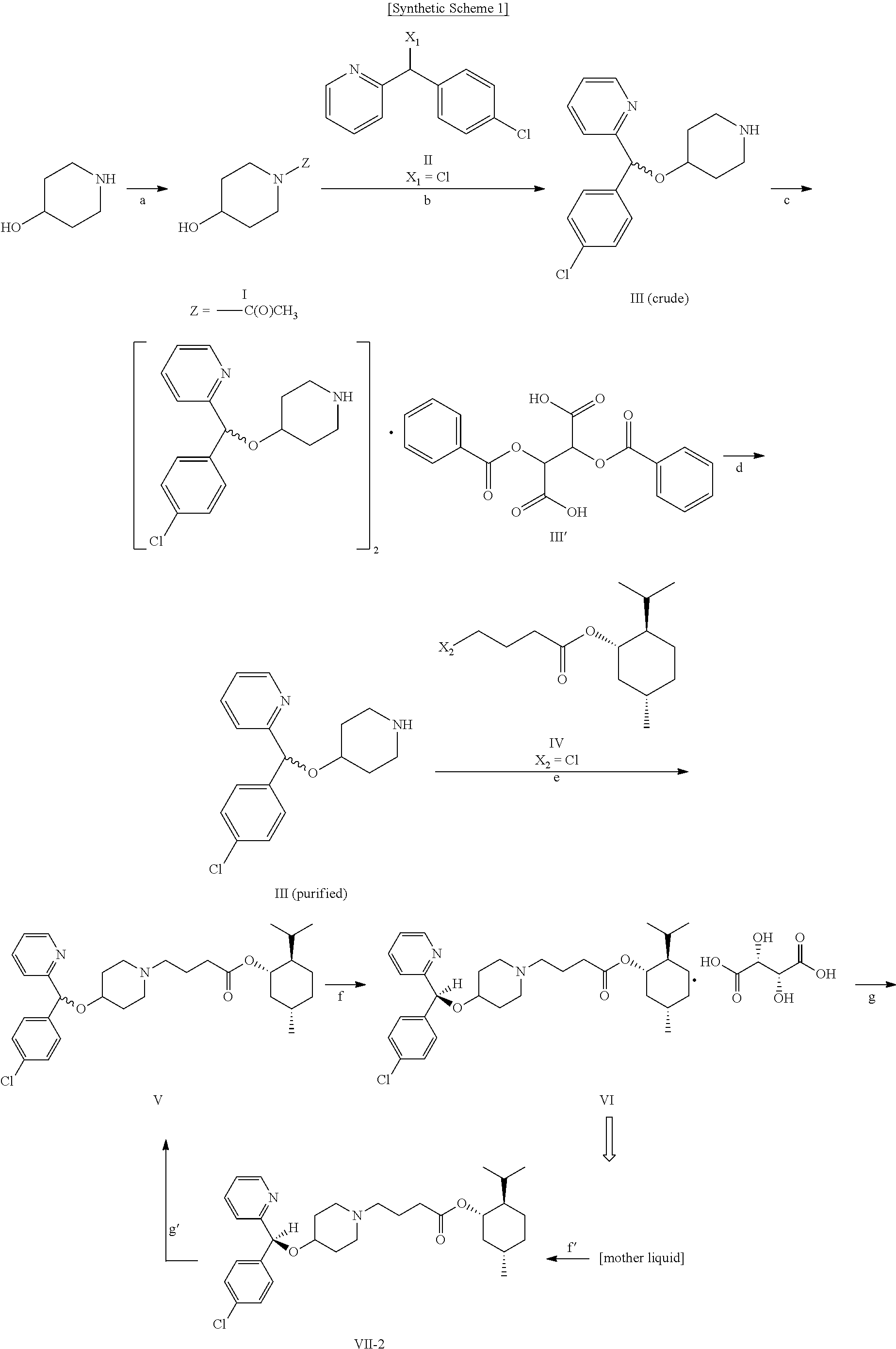

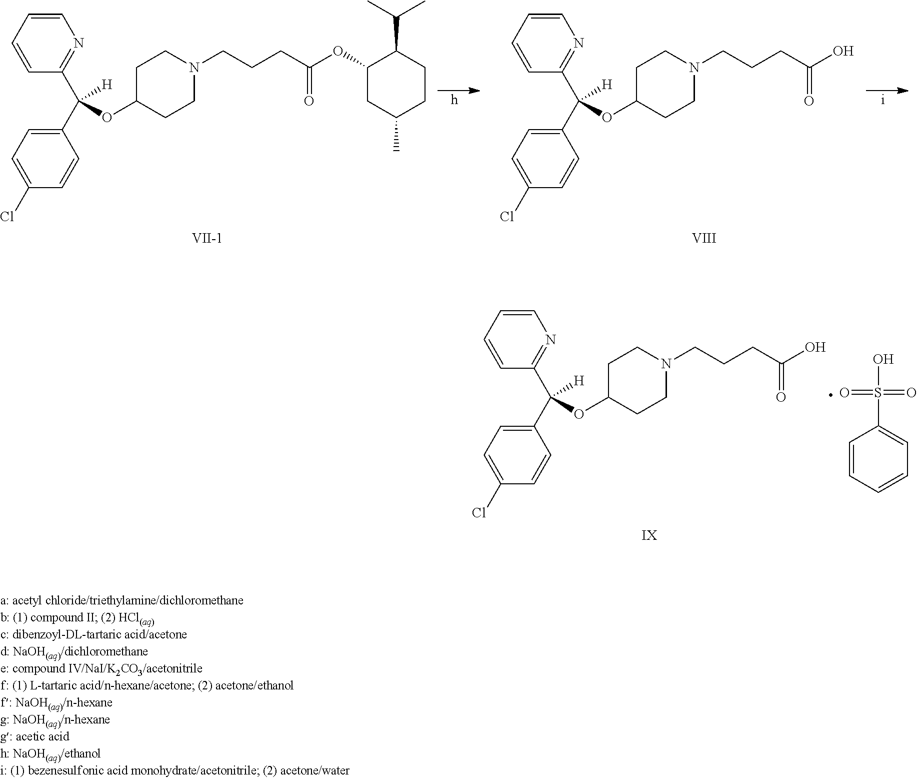

### Example 1

### Preparation of Compound (I) (Z═C(O)CH₃), N-Acetyl-4-hydroxypiperidine

4-hydroxypiperidine (1.0 kg) and triethylamine (1.2 kg) were added into a flask and dissolved in dichloromethane (20.0 kg), followed by adding acetyl chloride (0.8 kg) at −70° C. After 1 hour of stirring at −70° C., an aqueous solution of sodium hydroxide (2.0 kg, 25%) was added thereto, and the temperature was raised to room temperature. Finally, the solution was extracted with dichloromethane and evaporated under reduced pressure to get the compound (I) (Z=—C(O)CH₃, about 1.2 kg).

¹H NMR (CDCl₃) of Compound (I) (Z═—C(O)CH₃): δ 4.08 (m, 1H), 3.93 (m, 1H), 3.71 (m, 1H), 3.21 (m, 2H), 2.10 (s, 3H), 1.89 (m, 2H), 1.52 (m, 2H).

### Example 2

### Preparation of Compound (III), (RS)-4-[(4-chlorophenyl) (2-pyridyl) methoxy]piperidine)

The compounds (I) (1.2 kg) and (II) (1.0 kg, X₁═Cl, 2-[chloro(4-chlorophenyl)methyl]pyridine) were placed into a flask and reacted for 5 hours at 130° C., followed by adding HCl_{(aq)} (3.0 kg, 10%) and further 15 hours of reaction at 80° C. Next, the solution was cooled down to room temperature, alkalized with NaOH_{(aq)} (3.0 kg, 30%), extracted with dichloromethane, and then evaporated under reduced pressure to get the crude product (III) (about 940 g).

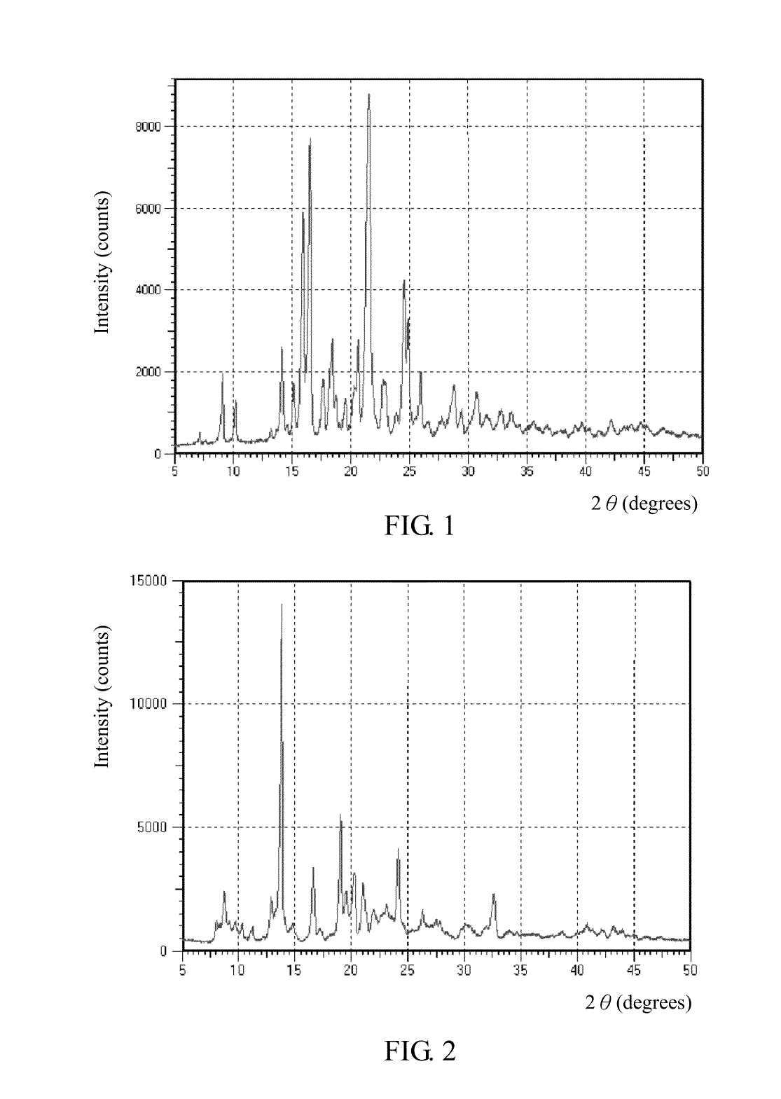

Dibenzoyl-DL-tartaric acid (560 g) was added into a flask and dissolved in acetone (6.0 kg), followed by adding the crude product (III) (940 g) thereto at 50° C. and then slow cooling to room temperature. After 16 hours of reaction at room temperature, the solution was filtered to get the salt compound (III′), bis-(RS)-4-[(4-chlorophenyl)(2-pyridyl)methoxy]piperidine 2,3-dibenzoyl-DL-tartrate. FIG. 1 shows its X-ray diffraction pattern.

Finally, the salt compound (III′) was mixed with NaOH_{(aq)} (5.5 kg, 10%), and the purified compound (III) (about 770 g) can be obtained by extraction with dicholormethane and solvent evaporation under reduced pressure. By high pressure liquid chromatography (HPLC) analysis, it can be determined that the purity of the compound (III) was enhanced from 86.01% to 97.33%.

¹H NMR (CDCl₃) of Compound (III′): δ 8.45 (m, 2H), 7.93 (m, 4H), 7.77 (m, 2H), 7.57 (m, 2H), 7.47 (m, 6H), 7.36 (s, 8H), 7.25 (m, 2H), 5.58 (s, 2H), 5.56 (s, 2H), 3.41 (m, 2H), 2.99 (m, 4H), 2.76 (m, 4H), 1.79 (m, 4H), 1.58 (m, 4H).

X-ray diffraction pattern of Compound (III′) having characteristic peaks at 2θ values about: 15.9, 16.5, 21.5, 24.6, 24.9 (±0.2).

¹H NMR (CDCl₃) of Compound (III): δ 8.51 (m, 1H), 7.68 (m, 1H), 7.53 (d, 1H), 7.37 (m, 2H), 7.28 (m, 2H), 7.16 (m, 1H), 5.63 (s, 1H), 3.53 (m, 1H), 3.10 (m, 2H), 2.61 (m, 2H), 1.92 (m, 2H), 1.58 (m, 2H).

### Example 3

### Preparation of Compound (V), (RS)-4-[4-[(4-chlorophenyl) (2-pyridyl)methoxy]piperidino]butyric acid D-menthyl ester

The compounds (III) (570 g), (IV) (860 g, X₂═Cl, D-menthyl-4-chlorobutanoate), sodium iodide (65 g), potassium carbonate (380 g) and acetonitrile (2.5 kg) were added into a flask and reacted for 15 hours at 80° C., followed by cooling to room temperature and then removing acetonitrile under reduced pressure. Next, water (1.2 kg) was added thereto, and the compound (V) (about 1.3 kg) was obtained by extraction with n-hexane and solvent evaporation under reduced pressure.

¹H NMR (CDCl₃) of Compound (V): δ 8.50 (m, 1H), 7.67 (m, 1H), 7.53 (d, 1H), 7.36 (m, 2H), 7.27 (m, 2H), 7.15 (m, 1H), 5.59 (s, 1H), 4.67 (m, 1H), 3.45 (m, 1H), 2.71 (m, 2H), 2.31 (m, 4H), 2.11 (m, 2H), 1.97 (m, 1H), 1.60-1.95 (m, 9H), 1.48 (m, 1H), 1.36 (m, 1H), 0.90-1.10 (m, 3H), 0.88 (d, 6H), 0.75 (d, 3H).

### Example 4

### Preparation of Compound (VI), (S)-4-{4-[(4-chlorophenyl) (2-pyridyl)methoxy]piperidino}butyric acid D-menthyl ester L-tartrate

The compound (V) (540 g), L-tartaric acid (160 g), acetone (1.3 kg) and n-hexane (1.1 kg) were added into a flask and stirred for 15 hours at room temperature followed by filtration to obtain solid product (690 g). Next, the solid product (690 g) was dissolved in a mixed solvent of acetone and ethanol under heating followed by slow cooling to 0° C. for crystallization. After 2 hours of crystallization at 0° C., the compound (VI) was isolated by filtration. Finally, about 180 g of the compound (VI) can be collected by repeating the above crystallization and filtration processes. FIG. 2 shows its X-ray diffraction pattern. Through HPLC analysis, the optical purity can be determined as 99.26% [(S)-isomer:(R)-isomer=99.26:0.74].

Melting point of Compound (VI): 151-154° C.

¹H NMR (CDCl₃) of Compound (VI): δ 8.54 (m, 1H), 7.71 (m, 1H), 7.40 (d, 1H), 7.35 (m, 2H), 7.31 (m, 2H), 7.22 (m, 1H), 5.59 (s, 1H), 4.66 (m, 1H), 4.30 (s, 2H), 3.79 (br, 1H), 3.30 (br, 2H), 3.15 (br, 2H), 3.01 (m, 2H), 2.39 (m, 2H), 2.16 (m, 2H), 2.04 (m, 4H), 1.94 (m, 1H), 1.78 (m, 1H), 1.67 (m, 2H), 1.47 (m, 1H), 1.34 (m, 1H), 0.90-1.10 (m, 3H), 0.89 (d, 6H), 0.73 (d, 3H).

X-ray diffraction pattern of Compound (VI) having characteristic peaks at 2θ values about: 13.8, 16.6, 19.0, 20.2, 24.1 (±0.2).

### Example 5

### Preparation of Compound (VII-1), (S)-4-[4-[(4-chloro phenyl)(2-pyridyl)methoxy]piperidino]]) butyric acid D-menthyl ester

The compound (VI) (180 g) was mixed with NaOH_{(aq)} (380 g, 10%), followed by extraction with n-hexane and solvent evaporation under reduced pressure to get the compound (VIM) (about 137 g).

¹H NMR (CDCl₃) of Compound (VII-1): δ 8.50 (m, 1H), 7.67 (m, 1H), 7.53 (d, 1H), 7.36 (m, 2H), 7.27 (m, 2H), 7.15 (m, 1H), 5.59 (s, 1H), 4.67 (m, 1H), 3.45 (m, 1H), 2.71 (m, 2H), 2.31 (m, 4H), 2.11 (m, 2H), 1.97 (m, 1H), 1.60-1.95 (m, 9H), 1.48 (m, 1H), 1.36 (m, 1H), 0.90-1.10 (m, 3H), 0.88 (d, 6H), 0.75 (d, 3H).

### Example 6

### Preparation of Compound (VIII), (+)-(S)-4-[4-[(4-chlorophenyl) (2-pyridyl)methoxy]piperidino]butyric acid

The compound (VII-1) (137 g), ethanol (500 g) and NaOH_{(aq)} (300 g, 10%) were added into a flask and reacted for 15 hours at room temperature, followed by ethanol removal under reduced pressure. Next, the solution was adjusted to pH 1.0 with 15% HCl_{(aq)}, washed with dichloromethane, adjusted to pH 6.0 with 30% NaOH_{(aq)}, extracted with dichloromethane, and then evaporated under reduced pressure to get the compound (VIII) (about 100 g).

¹H NMR (CDCl₃) of Compound (VIII): δ 8.53 (m, 1H), 7.70 (m, 1H), 7.43 (d, 1H), 7.34 (m, 2H), 7.29 (m, 2H), 7.20 (m, 1H), 5.56 (s, 1H), 3.76 (br, 1H), 3.07 (m, 4H), 2.90 (m, 2H), 2.51 (m, 2H), 2.16 (m, 2H), 2.02 (m, 4H)°

### Example 7

### Preparation of Compound (IX), (+)-(S-4-[4-[(4-chloro phenyl)(2-pyridyl)methoxy]piperidino]butyric acid monobenzene sulfonate

The compound (VIII) (100 g), benzenesulfonic acid monohydrate (43 g) and acetonitrile (450 g) were added into a flask and stirred for 15 hours at room temperature in the presence of seeds. Then, the solution was filtered to get the compound (IX) (about 118 g). Through HPLC analysis, the optical purity can be determined as 99.77% [(S)-isomer:(R)-isomer=99.77:0.23].

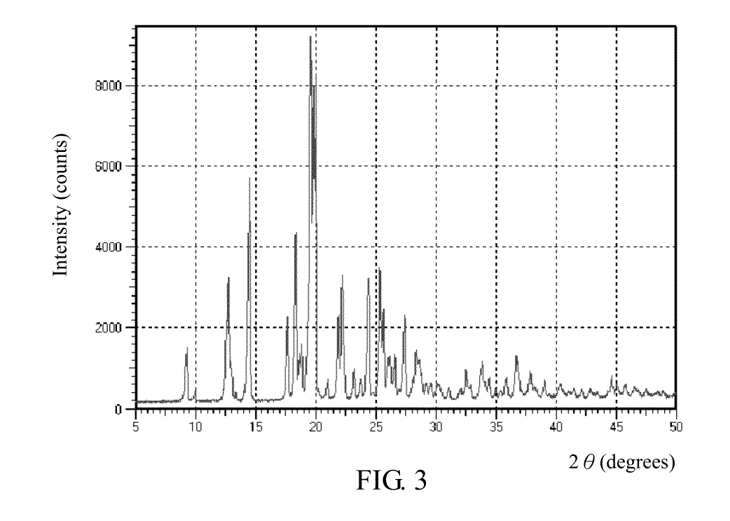

Next, the compound (IX) (118 g) was dissolved in acetone (380 g) and water (24 g) in a flask under heating, followed by adding seeds at 50° C. and slow cooling to 0° C. for crystallization. After 2 hours of crystallization at 0° C., the compound (IX) (about 96 g) was isolated by filtration. Through HPLC analysis, the optical purity can be determined as 99.93% [(S)-isomer:(R)-isomer=99.93:0.07]. FIG. 3 shows its X-ray diffraction pattern.

Melting point of Compound (IX): 161-166° C.

¹H NMR (DMSO-d⁶) of Compound (IX): δ 8.47 (m, 1H), 7.82 (m, 1H), 7.59 (m, 3H), 7.40 (m, 4H), 7.30 (m, 4H), 5.69 (s, 1H), 3.67 (br, 1H), 3.32 (br, 2H), 3.05 (br, 4H), 2.30 (t, 2H), 2.15 (br, 1H), 1.97 (br, 1H), 1.84 (m, 3H), 1.70 (br, 1H).

X-ray diffraction pattern of Compound (IX) having characteristic peaks at 2θ values about: 12.7, 14.5, 18.3, 19.6, 19.8, 22.2, 24.4, 25.4, 27.4 (±0.2).

### Example 8

### Recovering and Converting of Compound (VII-2), (R)-4-[4-[(4-chloro phenyl)(2-pyridyl)methoxy]piperidino]butyric acid D-menthyl ester

The crystal mother liquor of the crystal (VI) was concentrated to remove solvent, followed by adding 10% NaOH_{(aq)} (1.2 kg), extraction with n-hexane and evaporation to get the compound (VII-2) (about 320 g).

The compound (VII-2) (320 g) [(S)-isomer:(R)-isomer=29.80:70.20] and acetic acid (320 g) were added into a flask and reacted for 6 hours at 110° C. After the reaction is accomplished, water (850 g) and 30% NaOH_{(aq)} (850 g) were added thereto, and the solution was extracted with n-hexane and evaporated to get the compound (V) (about 308 g). Through HPLC analysis, the ratio of (S)-isomer:(R)-isomer can be determined as 49.79:50.21.

¹H NMR (CDCl₃) of Compound (VII-2): δ 8.50 (m, 1H), 7.67 (m, 1H), 7.53 (d, 1H), 7.36 (m, 2H), 7.27 (m, 2H), 7.15 (m, 1H), 5.59 (s, 1H), 4.67 (m, 1H), 3.45 (m, 1H), 2.71 (m, 2H), 2.31 (m, 4H), 2.11 (m, 2H), 1.97 (m, 1H), 1.60-1.95 (m, 9H), 1.48 (m, 1H), 1.36 (m, 1H), 0.90-1.10 (m, 3H), 0.88 (d, 6H), 0.75 (d, 3H).

Although the present invention has been explained in relation to its preferred embodiment, it is to be understood that many other possible modifications and variations can be made without departing from the spirit and scope of the invention as hereinafter claimed.

What is claimed is:

1. A method of synthesizing bepotastine or benzenesulfonic acid salt thereof, comprising:
(a) providing a compound represented by the following formula (V), 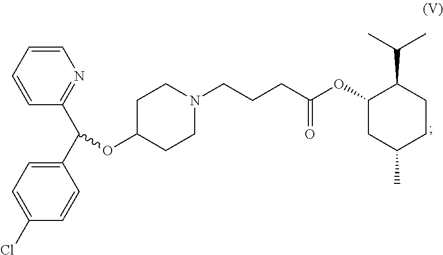 [SMILES: CC(C)[C@H]1CC[C@H](C)C[C@@H]1OC(=O)CCCN1CCC(OC(c2ccc(Cl)cc2)c2ccccn2)CC1]
(b) treating the compound of the formula (V) with L-α-hydroxy acid to conduct optical resolution process and isolating an L-α-hydroxy acid salt formed of a compound represented by the following formula (VII-1) and the L-α-hydroxy acid, 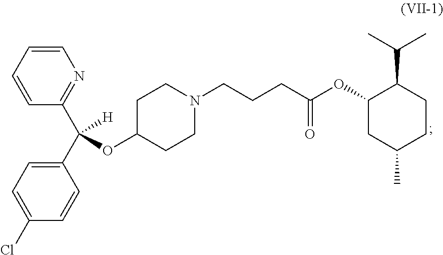 [SMILES: CC(C)[C@H]1CC[C@H](C)C[C@@H]1OC(=O)CCCN1CCC(O[C@@H](c2ccc(Cl)cc2)c2ccccn2)CC1]
and
(c) subjecting the L-α-hydroxy acid salt to acid removal and ester hydrolysis treatment to produce a compound represented by the following formula (VIII) or further to produce benzenesulfonic acid salt thereof,

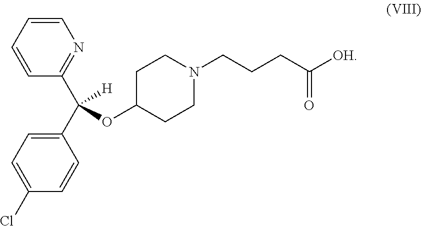 [SMILES: O=C(O)CCCN1CCC(O[C@@H](c2ccc(Cl)cc2)c2ccccn2)CC1]

2. The method as claimed in claim 1, wherein the L-α-hydroxy acid is L-tartaric acid, and the L-α-hydroxy acid salt is a salt compound represented by the following formula (VI),

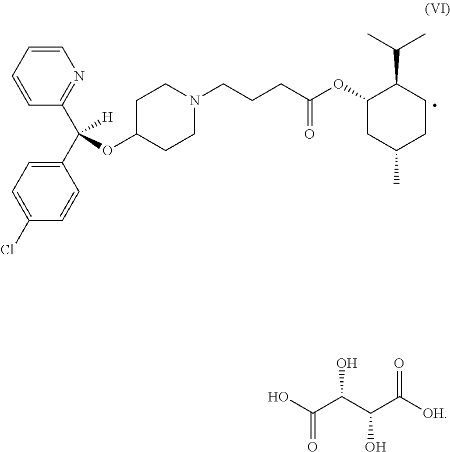 [SMILES: CC(C)[C@H]1CC[C@H](C)C[C@@H]1OC(=O)CCCN1CCC(O[C@@H](c2ccc(Cl)cc2)c2ccccn2)CC1 ; O=C(O)[C@H](O)[C@@H](O)C(=O)O]

3. The method as claimed in claim 1, wherein the compound of the formula (V) is prepared by the following step:
subjecting a compound represented by the following formula (III) and a compound represented by the following formula (IV) to substitution reaction, 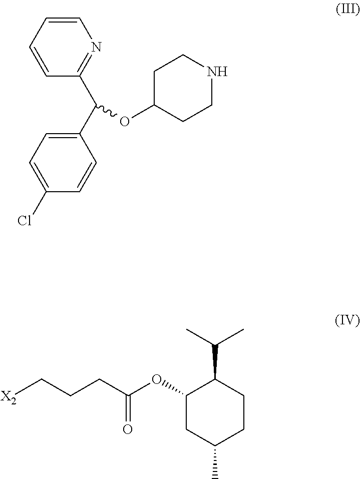
wherein X₂ is a leaving group.

4. The method as claimed in claim 3, wherein the compound of the formula (III) is prepared by the following steps:
subjecting a compound represented by the following formula (I) and a compound represented by the following formula (II) to substitution reaction and then deprotection reaction, 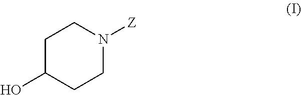 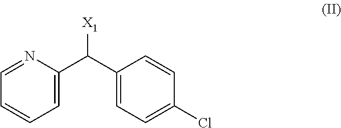
wherein Z is a protecting group for amino group, and X₁ is a leaving group.

5. The method as claimed in claim 3, wherein the compound of the formula (III) is purified before being subjected to the reaction of producing the compound of the formula (V) by the following steps:
producing and isolating a salt compound represented by the following formula (III′) using dibenzoyl-DL-tartaric acid, and then conducting acid removal treatment to liberate the compound of the formula (III),

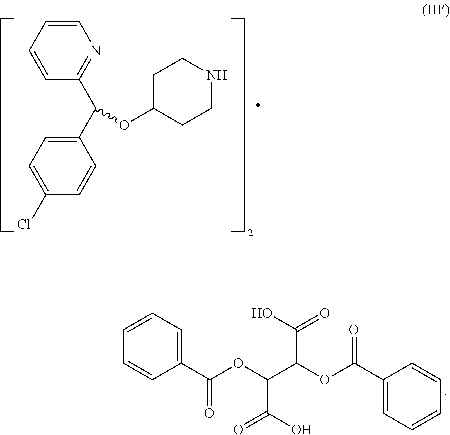

6. The method as claimed in claim 1, further comprising:
recovering a compound of the following formula (VII-2) which is excluded in the optical resolution process, and converting the compound of the formula (VII-2) into the compound of the formula (V),

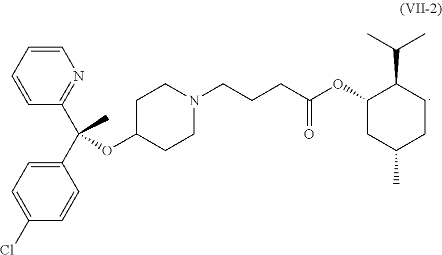 [SMILES: CC(C)[C@H]1CC[C@H](C)C[C@@H]1OC(=O)CCCN1CCC(O[C@](C)(c2ccc(Cl)cc2)c2ccccn2)CC1]

7. The method as claimed in claim 4, wherein Z is acetyl, and X₁ is halogen.

8. The method as claimed in claim 3, wherein X₂ is halogen.

9. The method as claimed in claim 6, wherein the compound of the formula (VII-2) is converted into the compound of the formula (V) by an acid.

10. A compound represented by the following formula (VII-1) or its L-α-hydroxy acid salt,

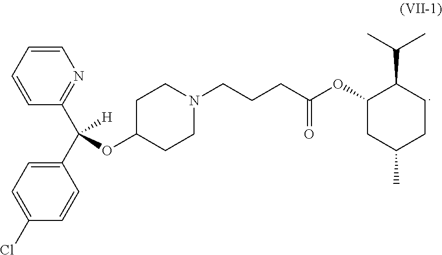 [SMILES: CC(C)[C@H]1CC[C@H](C)C[C@@H]1OC(=O)CCCN1CCC(O[C@@H](c2ccc(Cl)cc2)c2ccccn2)CC1]

11. The compound or its L-α-hydroxy acid salt as claimed in claim 10, wherein the L-α-hydroxy acid salt is a salt compound represented by the following formula (VI), 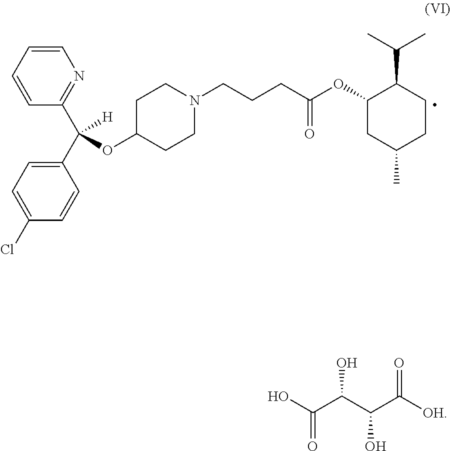 [SMILES: CC(C)[C@H]1CC[C@H](C)C[C@@H]1OC(=O)CCCN1CCC(O[C@@H](c2ccc(Cl)cc2)c2ccccn2)CC1 ; O=C(O)[C@H](O)[C@@H](O)C(=O)O]
